(19) United States
(12) Patent Application Publication
(10) Pub. No.: US 2024/0315366 A1
(43) Pub. Date: Sep. 26, 2024

(54) AN APPARATUS AND METHOD FOR CUSTOMIZING THE FIT OF A FACE MASK

(71) Applicant: Impressive Engineering Limited, Aberdeen (GB)

(72) Inventor: Kasra Zarisfi, Aberdeen (GB)

(21) Appl. No.: 18/272,743

(22) PCT Filed: Jan. 31, 2022
(86) PCT No.: PCT/GB2022/000013

§ 371 Date: Jul. 17, 2023

(30) Foreign Application Priority Data
Feb. 2, 2021 (GB) 2101400.6
Feb. 24, 2021 (GB) 2102647.1
Jan. 29, 2022 (GB) 2201177.9

(51) Int. Cl. A41D 13/11

(52) U.S. Cl. CPC A41D 13/1161

(57) ABSTRACT

A method of forming a belt for a face mask, comprising the steps of: using a measuring means to trace the contours of the face of a user, and using the data collected by the measuring means to create a cut line on a loop of material; wherein the step of using a measuring means to trace the contours of the face of a user by an electronic measuring tool, a software program and the data collected by the measuring means can be used to create a 3D model of the face as a mathematical representation, and extract a closed 3D curve from the 3D model.

### REFERENCE TO PRIOR APPLICATION

This application is a national stage application of PCT application PCT/GB2022/000013 filed on Jan. 31, 2022 and claiming priority of Feb. 2, 2021 entitled AN APPARATUS AND METHOD FOR CUSTOMIZING THE FIT OF A FACE MASK, by inventor Kasra Zarisfi and Applicant Impressive Engineering Limited.

### FIELD

The present invention relates to an apparatus for customising the fit of a face mask.

The present invention also relates to a method for customising the fit of a face mask.

### BACKGROUND

Face masks are used in different shapes and forms for different purposes. For example, a face mask can be shaped and sized so as to form a sealed or semi-sealed volume around the nose and mouth of a user, so as to assist with controlling the air/oxygen supply, or to provide an area into which filtered or purified air is delivered, with unwanted particles such as dust, pollen, soot, smoke, or liquid droplets, or other items such as microbes, viruses, bacteria, etc, removed from the air delivered to the mask.

Face masks of this type cover at least part of the user's face—the mouth, or the nose, or the nose and mouth, or the full face—and usually comprise a rigid or semi-rigid body, fitted with a flexible perimeter seal that seals against the surface of the user's face, to prevent the uncontrolled entry of external air into the internal volume of the mask.

However, because people's faces come in different shapes and sizes, it is difficult to design a ‘one size fits all’ face mask which will provide a good fit for everyone. Face masks are mass-produced, and this requires a single, fixed shape product (mask), or a limited range of sizes.

Because of this, any one mask will not necessarily be matched to the face of a particular user, and this may cause issues with the seal. Any difference between the contours of the face and the contours of the section of the mask which in contact with the face can considerably affect the performance of the mask and can potentially result in the mask not sealing against the face of a user.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY

It is an object of the present invention to provide an apparatus for customising the fit of a face mask which goes some way to overcoming the abovementioned disadvantages or which at least provides the public, health care, or industry with a useful choice.

It is a further object of the invention to provide a method for customising the fit of a face mask which goes some way to overcoming the abovementioned disadvantages or which at least provides the public, health care, or industry with a useful choice.

The term “comprising” as used in this specification and indicative independent claims means “consisting at least in part of”. When interpreting each statement in this specification and indicative independent claims that includes the term “comprising”, features other than that or those prefaced by the term may also be present. Related terms such as “comprise” and “comprises” are to be interpreted in the same manner.

As used herein the term “and/or” means “and” or “or”, or both. As used herein “(s)” following a noun means the plural and/or singular forms of the noun.

Accordingly, in a first aspect the present invention may broadly be said to consist in a method of forming a belt for a face mask, comprising the steps of: i) using a measuring means to trace the contours of the face of a user, and; ii) using the data collected by the measuring means to create a cut line on a loop of material, and; iii) cutting the belt along the cutline, and; iv) connecting the belt to a face mask body so that the belt forms a seal for the mask.

In an embodiment, in the step of using a measuring means to trace the contours of the face of a user, step i), a user positions patterned stickers on his/her face and take a photo from the front of the face.

In an embodiment, the method comprises the additional step between steps i) and ii) a software program is used to analyse the data.

In an embodiment, in the step of using a software program, the stickers' patterns are extracted from the photo.

In an embodiment, in the step of using a software program, the difference between the flat stickers' patterns and the stickers' patterns from the photo are analysed to define a mathematical representation of a closed 3D curve on the face.

In an embodiment, in the step of using a software program, the closed 3D curve is mathematically converted to a 2d open curve.

In an embodiment, in the step of using a software program, the defined 2d open curve is converted to a graphical image of a 2D shape and be sent to the user.

In an embodiment, in the step of using the data collected by the measuring means to create a cut line on a loop of material, step ii) user prints the image of the 2D shape on a paper and cut it out along its perimeter.

In an embodiment, in the step of using the data collected by the measuring means to create a cut line on a loop of material, step ii), the user wraps the 2D shape around the belt and use it as a guide for manual cutting of the belt.

In an embodiment, in the step of using a measuring means to trace the contours of the face of a user, step i), an electronic measuring tool is used.

In an embodiment, the method comprises the additional step between steps i) and ii) of using the data collected by the measuring means to create a 3D model of the face as a mathematical representation, and extracting a closed 3D curve from the 3D model.

In an embodiment, in the step iii) the method comprises placing a belt on a cutting machine and transferring data to the cutting machine, so that the cutting machine cuts the belt in accordance with the 3D curve.

In a second aspect the present invention may broadly be said to consist in a belt for a face mask, comprising: a loop of soft material suitable for extended skin contact and configured to connect to a mask body in use, the loop formed with a profile that generally matches that of the facial features in the location in which the face mask is to be used, the loop and mask body mutually configured to form a closed volume in front of at least a portion of the face of a user, the loop in use locating against the face of a user to form a seal; the belt formed from the loop using the method of any one of the preceding statements.

In a third aspect the present invention may broadly be said to consist in an apparatus for customising the fit of a face mask, comprising: a bar support structure configured to form a closed or substantially closed loop; a plurality of elongate bars, the bars and bar support structure configured so that the bars are held in place on the bar support structure adjacent to one another around the closed loop; the bar support structure further configured so that each of the individual bars can slide within the support structure in a direction substantially parallel to the longitudinal axis of the bar, at least one end of each bar protruding freely when the bar is extended within the support structure; the closed loop of the bars sized and configured so as to form a loop of substantially equivalent size to the perimeter of the face mask's belt.

In an embodiment, the bar support structure and bars are further configured so that there is sufficient friction between the bars and the support structure to retain the bars in a particular position once they are pushed to that position.

In an embodiment, the bars and support structure are configured so that the bars can displace a distance of substantially 50 mm from a starting position where a bar is substantially fully extended away from the central ring of the support structure, to a position where the bar has been pushed fully towards the support structure.

In an embodiment, the bars are arranged substantially in parallel with one another.

In an embodiment, each of the bars comprises an elongate body and head, the head wider than the body, the bars and support structure configured so that the heads are substantially directly adjacent to one another.

In an embodiment, the apparatus for customising the fit of a face further comprises a belt, the belt comprising a continuous closed loop of soft material suitable for extended skin contact, the belt and bar support structure mutually configured so that the belt can in use locate onto the bar support structure so the bars slide substantially parallel to and directly adjacent to the outer surface of the belt.

In an embodiment, the belt is formed from neoprene, silicone rubber, or any other suitable elastic or soft polymer.

In an embodiment, the width of the belt is sufficient to allow cutting to the required shape for substantially all face shapes and sizes.

In an embodiment, the belt has a minimum width of 5 mm at any point along the belt.

In an embodiment, the belt is formed with a profile that generally matches that of the facial features in the location in which the belt is to be used.

In an embodiment, the belt further comprises at least one connector configured to connect the belt to a mask body.

In an embodiment, the at least one connector comprises a female connector.

In an embodiment, the connector comprises a tongue and groove connection.

In a fourth aspect the present invention may broadly be said to consist in a method of forming a belt for a face mask, comprising the steps of: i) placing an apparatus as against the face of a user, with the bars substantially fully extended; ii) pressing the apparatus against the face of a user so that the bars slide inwards to form an overall shape that conforms to the contours of a user's face around a closed loop in the required location; iii) positioning a belt comprising a continuous closed loop of soft material suitable for extended skin contact within the closed loop of the bars; iv) tracing a line around the outer ends of the bars on the outer surface of the belt; v) removing the belt and cutting along the traced line; vi) connecting the belt to a face mask body so that the belt forms a seal for the mask.

In an embodiment, the method of forming a belt for a face mask comprises the further step between steps iv) and v) of wrapping a strap or similar around the bars to hold these in place.

In a fifth aspect the present invention may broadly be said to consist in belt for a face mask, comprising: a loop of soft material suitable for extended skin contact and configured to connect to a mask body in use, the loop formed with a profile that generally matches that of the facial features in the location in which the face mask is to be used, the loop and mask body mutually configured to form a closed volume in front of at least a portion of the face of a user, the loop in use locating against the face of a user to form a seal, the belt formed from the loop using the method of the preceding statements.

With respect to the above description then, it is to be realised that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

### BRIEF DESCRIPTION OF THE FIGURES

Further aspects of the invention will become apparent from the following description which is given by way of example only and with reference to the accompanying drawings which show an embodiment of the device by way of example, and in which:

FIG. 1 shows a perspective view from above and to one side of an embodiment of a mask body that forms part of the apparatus of the present invention, the body shown in simplified form for ease, the mask body configured to in use cover part of the face, such as the mouth and nose, the mask body having a front face and a surrounding side wall, the side wall containing filter areas, the side wall of the mask body having indented sections on its outer rim that extend towards the front face.

FIG. 2 shows a perspective side view from another angle of the body of FIG. 1.

FIG. 3 shows a perspective view from one side and below of the body of FIGS. 1 and 2.

FIG. 4 shows a perspective view from one side and above of a form of belt that is used with the body of FIGS. 1 to 3, the belt configured to in use form a perimeter seal on the face of a user, the belt formed of a soft, flexible and compressible material, and having an upper or front edge that conforms with and is matched to the contours of the rear edge of the body of FIGS. 1 to 3, the belt attaching via it's front edge to the rear edge of the wall of the body.

FIG. 5 shows a perspective side view from another angle of the belt of FIG. 4.

FIG. 6 shows the belt of FIGS. 4 and 5, with cut-out sections on the back rim or rear edge formed to match the shape of the face of a user.

FIG. 7 shows a perspective view from one side and above of the body and belt of the preceding figures assembled together.

FIG. 8 shows a perspective view from one side and below of the body and belt of the preceding figures assembled together.

FIG. 9 shows a measuring tool configured for measuring the face curvature of a user when wearing a mask, the measuring tool comprising a bar support structure that is in use configured to hold a number of bars, which can be pushed to a position so that the pattern of the bars conforms to the profile of a user's face around a closed perimeter, the bars not shown for clarity, the measuring tool having a body comprising a sleeve, into which a belt can fit.

FIG. 10 shows the measuring tool of FIG. 9, with all the bars fully shown, and fully extended.

FIG. 11 shows the measuring tool of FIGS. 9 and 10 with the bars shown pushed to a position so that the pattern of the bars conforms to the profile of a user's face around a closed perimeter.

FIG. 12 shows the measuring tool of FIG. 11 with a belt inserted into the closed perimeter formed by the bars, the bars marking on the external surface of the belt where the belt can be cut, so that the edge profile of the belt fits the users face.

FIG. 13 shows an electronic measuring tool in use, the electronic measuring tool used to measure the profile of a user's face.

FIG. 14 shows stickers with patterns on the face for taking a photo and a software program in use to measure the profile of a user's face.

FIG. 15 shows a 2D shape printed on a paper which has a matching curve with the face. The 2D shape can be cut and be wrapped around the belt as a guide for cutting the belt.

FIG. 16 shows air flow diagram through the body of the mask.

### DETAILED DESCRIPTION

Embodiments of the invention, and variations thereof, will now be described in detail with reference to the figures.

An embodiment of apparatus for customising the fit of a face mask is shown in the figures. The apparatus of the preferred embodiment comprises three main parts: a body 1, a belt 5, and a measuring tool 10.

### Body

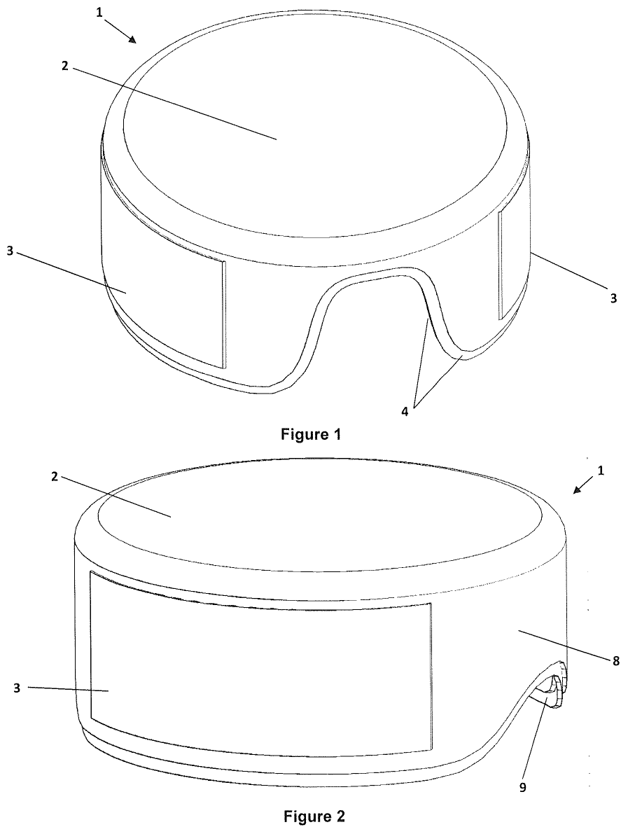

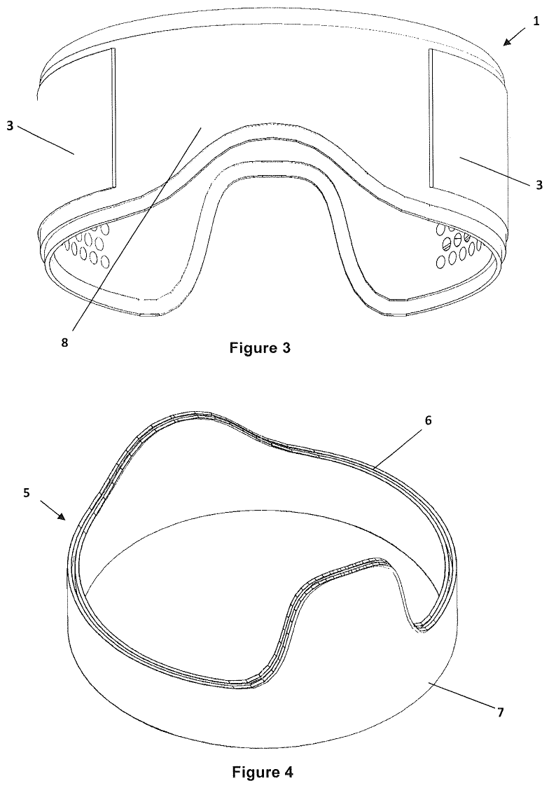

The body forms the rigid or semi-rigid body of a face mask. As shown in FIGS. 1 and 2, in the simplified embodiment shown, the body 1 comprises a substantially flat front face portion 2, with a surrounding wall 8 extending from the edge of the front face portion 2. In use, the body 1 is secured to the face of a wearer/user via straps or similar (not shown). The body can be provided in a variety of different shapes, suitable for a full-face fit, a fit around the nose-and-mouth, a fit around the nose only or any other type of mask fit.

In preferred embodiments, the body 1 is formed partly or wholly from plastic and/or polymer material, and can be washed or sterilised. The front face portion 2 can be formed of a transparent plastic or polymer, to allow the face of the wearer to be seen through the front face. This helps with communication and body language since the persons' mouth can be seen by others when talking. For consistency and ease of manufacturing, the whole body can be made from a transparent plastic or polymer.

Filter sections 3 are formed in the wall 8, to allow air to enter the closed space formed by the wall 8, the front face portion 2, and the seal formed by belt 5 (as described below), and the face of a user when the mask is worn. The filter sections 3 comprise holes or gaps in the wall 8, with the wall 8 adapted in these areas for the attachment of filters or filter material so that air entering the body 1 through the filter sections 3 has to pass therethrough. It should be noted that in other embodiments, the filter sections could be formed as attachment points for hoses or similar, so that clean air or oxygen can be provided to the mask from a remote location. ‘Filter section’ as used in this specification should be taken as broad enough to cover both of these options, and all similar options.

One advantage of having the filter sections 3 in the wall 8, rather than at the front (e.g. on the front face), is that the face of a user can be seen, if the front face portion 2 is transparent.

The space inside the body is separated in 3 chambers.
1—Nose and mouth chamber 20, is a space in front of nose and mouth. The inhale and exhale gases are mixed in this chamber.
2—Inhale chamber 21, is a space for inhale gases. There is a one-way valve 23 between the Inhale chamber and the Nose and mouth chamber which only allows the gases to flow from the Inhale chamber to the Nose and mouth chamber and not in the opposite direction. The outside air is purified by a filter 3 before entering the Inhale chamber for breathing.
3—Exhale chamber 22, is a space for exhale gases. There is a one-way valve 23 between the Nose and mouth chamber and the Exhale chamber which only allows the gases to flow from the Nose and mouth chamber to the Exhale chamber and not in the opposite direction. The exhale gases can be cleaned by a filter 3 before leaving the mask or can be directly sent to the atmosphere without filtering.

The wall 8 has a rear edge 4, on the side away from the front face portion 2. The rear edge 4 is generally shaped with indents or similar that generally correspond to the features of a generic user's face, such as their nose, chin, or similar. The rear edge 4 is in the preferred embodiment is fitted with male connector parts 9, or is itself at least partly formed as a male connector part, for connecting the belt 5 as described below. The male connector parts 9 can form a tongue for a tongue and groove connection between the body and the belt.

### Belt

As shown in FIGS. 4 and 5, in one embodiment, the belt 5 forms a continuous closed loop of substantially the same size (perimeter) as the rear edge 4 of the wall 8. The belt 5 is in use connected to the body 1 so that the belt 5 extends from the rear edge 4 of the wall 8 towards the face of a wearer. The purpose of the belt 5 is to act as a spacer or a seal between the body 1 and the face of the wearer. The belt 5 is in contact with the skin and spreads the pressure from wearing the mask over the face. The belt 5 is formed with female connectors corresponding to the male connector parts 9 of the body, in order to connect the belt 5 and body 1.

The belt 5 in the preferred embodiment is formed of a soft material suitable for extended skin contact, such as for example neoprene, silicone rubber or any other suitable elastic or soft polymer.

The profile of the front edge 6 of the belt 5—towards the body 1—is formed so as to substantially match the rear edge 4 of the wall 8 of the body 1. The belt 5 is formed so that the male connector parts 9 can attach to corresponding female parts on the belt 5 to connect the belt 5 and body 1 together.

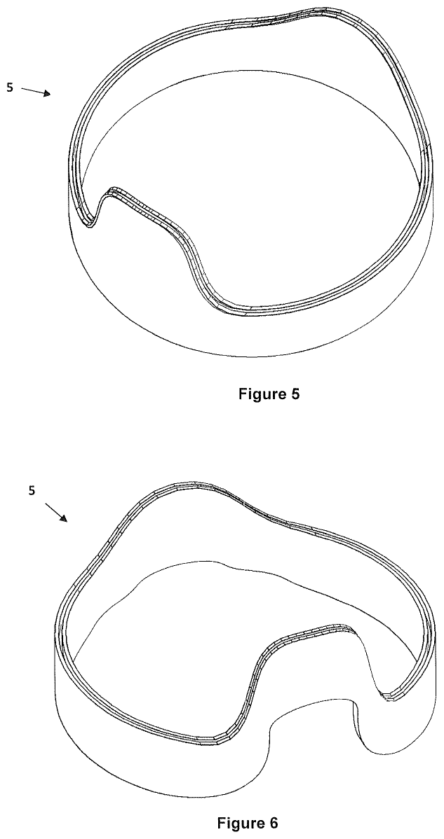

As shown in the embodiment of FIGS. 4 and 5, the rear edge 7 of the belt 5 is of a uniform height, with no indents or similar. It is intended that the rear edge 7 can be cut by a user to match the curvature and contour of their own face. Therefore, the width of the belt (between the front and rear edges) is such that it is wide enough to allow cutting to the required shape, for substantially all face shapes and sizes. After cutting the belt a minimum width of 5 mm at any point along the belt 5 is preferred to be left. In some embodiments, the belt 5 is formed so as to be wider where there are cutouts in the body 1 for the nose and chin and other facial features, so that the belt will fill the cutouts, as shown in FIG. 5. The belt 5 in these areas is wide enough to allow cutting to the shape of a user's face as shown in FIG. 6.

It is also preferred that the belt has a minimum thickness (the dimension roughly aligned in the same plane as the flat face portion 2 in use) of substantially 3 mm. That is, the belt is 3 mm thick where it is in contact with the face of a wearer.

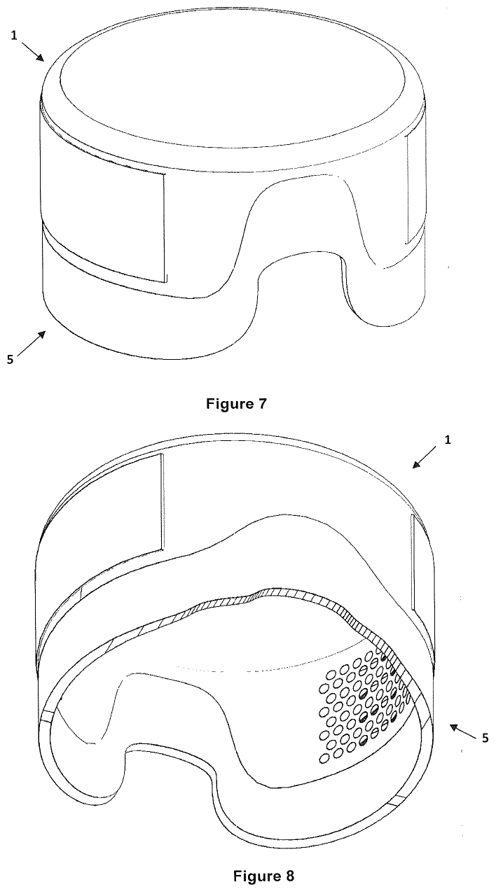

As shown in FIGS. 7 and 8, the body 1 and belt 5 are, in the preferred embodiment, push- or friction-fitted together via the male connector parts 9 and corresponding female connector parts, such as a tongue and groove connection, with the tolerances between the belt 5 and body 1 formed such that the fit between the two is watertight and airtight, but so that the two can be pulled apart by hand.

### Measuring Tool

A measuring tool 10 is used to measure and trace the outline of the face of an individual user, so that the user can shape the mask to their individual features, for improved fit and comfort.

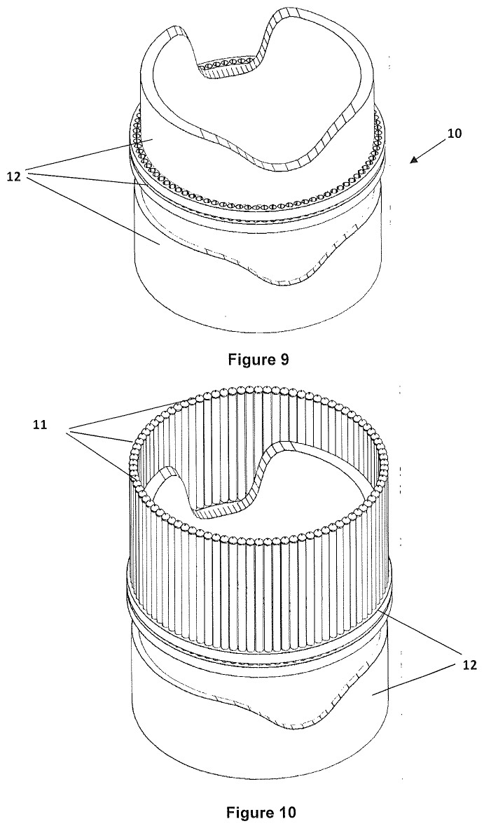

The measuring tool 10 is shown in FIGS. 9 to 12. The measuring tool 10 comprises a number of bars 11 located directly next to one another, and a bar support structure 12 that holds the bars 11 in place on the bar support structure, to form a closed loop, with each bar directly adjacent to and parallel to it's immediate neighbours. The bar support structure 12 is further configured so that each of the individual bars 11 can slide within the support structure 12 in a direction parallel to it's longitudinal axis. The support structure 12 and bars 11 are configured so that the bars 11 can slide freely within the support structure 12, but so that there is sufficient friction between the bars 11 and the support structure 12 to retain the bars 11 in a particular position once they are pushed to that position.

In a preferred embodiment, the bars 11 and support structure 12 are configured so that the bars 11 can displace a distance of substantially 50 mm from a starting position where a bar 11 is fully extended away from the central ring of the support structure 12, to a position where the bar 11 has been pushed fully towards the support structure 12.

In a preferred embodiment, each of the bars comprises an elongate body, with a wider head. This ensures that the body of the bar 11 can locate into the support structure in a discrete location, physically separated from the remaining bars 11 by the body of the support structure 12, but also so that the heads will be substantially directly adjacent to one another. This helps to ensure that the coverage of the bars 11 where they are in contact with the face of a user (as explained below) is substantially continuous.

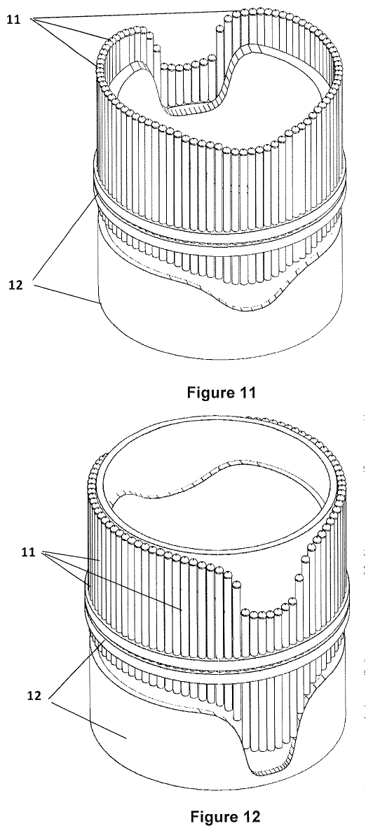

The bars 11 are shown fully extended in their starting position in FIG. 10.

In use, a user will start the measuring process with all of the bars 11 in this position. The user will then gently place their face in the centre of the assembly as it is shown in FIG. 10 for a preferred embodiment, and push against this (or hold this up to push this against their face). In doing so, the bars 11 are pushed inwards, so that the pattern of the tips of the bars 11 as a whole mirrors the contours of the face of the user in the locations where the heads of the bars 11 have contacted the face.

FIG. 11 shows the position of the bars 11 after a user has carried out this process. As outlined above, the friction between the bars 11 and the support structure 12 is such that the bars 11 will remain in the position to which they are pushed, unless force is applied directly, to move them out of this position. However, if required a strap, or elastic band, or similar can be wrapped around the bars 11 to ensure that they remain in position.

As shown in FIG. 12, the measuring tool 10 has a central cavity or sleeve, into which a belt 5 can be placed, inside the ring formed by the bars 11. The profile of the outer edge of the sleeve is formed so as to substantially match the front edge 6 of the belt 5.

As shown in FIG. 12, the belt 5 extends upwards inside and around the face profile formed by the bars 11. A user can mark the belt with their face profile by tracing around the belt surface just above the ends of the bars 11.

A user can then remove the marked-up belt 5 from the measuring device 10, and cut along the marked-up line, in order to produce a belt 5 that substantially exactly matches their own face profile. It is preferred that when this process is carried out, the belt 5 has a minimum width (under the top of the bars 11) of at least 5 mm at every point, so that after the cutting operation is carried out, the belt will have a minimum width of 5 mm at any point.

The measuring tool can be used by different users, each with a different face profile, with each individual user resetting the bars (pulling them upwards/outwards to their greatest extent), profiling their own face, and then cutting a new belt to fit that profile, as outlined above.

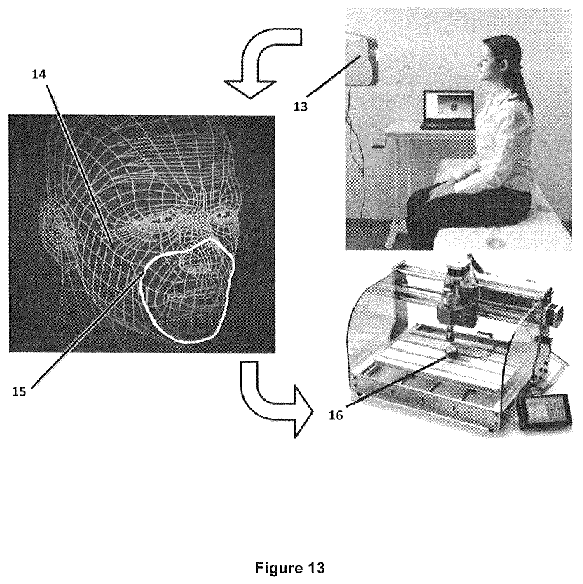

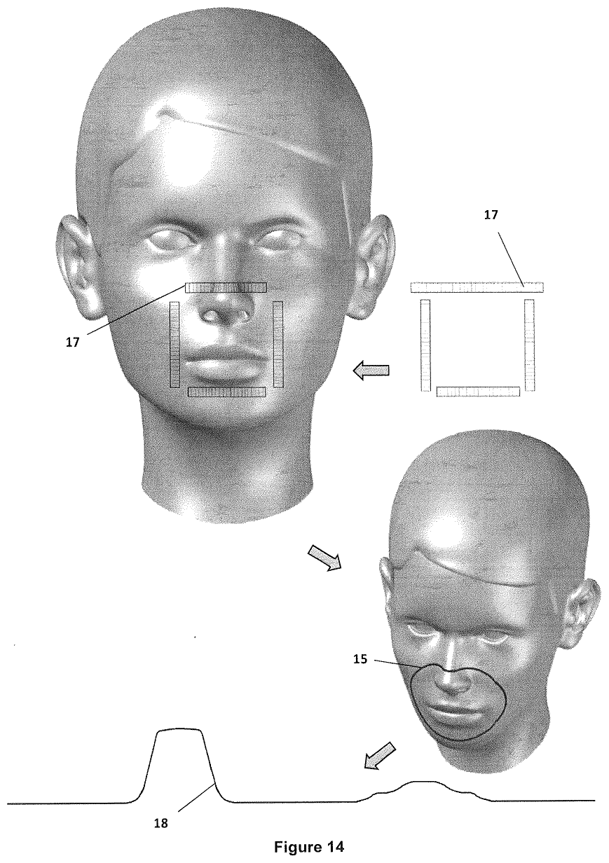

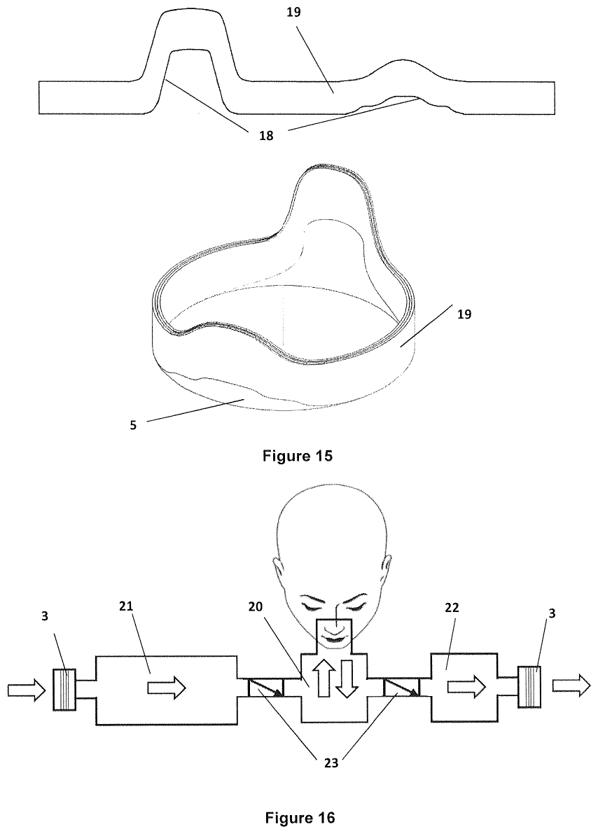

In an alternative embodiment, an electronic measuring tool can be used instead of the arrangement described above, for measuring the face curvature of a mask. In this embodiment, and as shown in FIG. 13, a 3D scanner 13 scans an individual's face to measure the face curvatures and profile. The scanner creates a 3D model of the face as a mathematical representation 14, using 3D graphic software or modelling software. The relevant geometry of a closed 3D curve 15 (that is, the line on the face on which the mask will sit) is extracted from the face model. A belt 5 is then placed in a cutting machine 16 and the data is transferred to the cutter. The cutter 16 cuts the belt as per the 3D curve geometry to create a belt that is matched with the face of the individual.

Alternatively, as shown in FIG. 14, the mask provider may supply stickers 17 marked with specific patterns like strips for the customers. The individual should stick these stickers 17 on his/her face while taking a photo. A photo should be taken from the front.

A software program is used to do the followings:
i) The stickers' patterns are extracted from the photo;
ii) The stickers on the face have different pattern from the flat stickers. For example, the distances between the stripes are different and some of the lines are changed to curves.

The difference between the patterns of the flat stickers and the stickers on the face are analysed to define a mathematical representation of a closed 3D curve on the face 15;
iii) The closed 3D curve on the face 15 then is mathematically converted to a 2d open curve of the face 18;
iv) The defined 2d open curve 18 is converted to a graphical image of a 2D Shape 19.
   The bottom curve of the 2D shape 18 is match with the 2D curve of the face 18;
   The top curve of the 2D shape is a 2D curve which is extracted from the belt's front edge 6;
   The length of the 2D shape 19 is the same as the circumference of the belt 5.
v) The 2D Shape 19 as an graphical image is sent to individuals, so they can print it on paper.

The printed 2D Shape on the paper (flat) 19 should be cut out around its circumference and be wrapped around the belt 5. The individual can use the wrapped 2D Shape 19 as a guide to cut the belt 5.

The apparatus and method as described above in the preferred embodiment are for a mask of the type that covers the nose and mouth of a user, but which leaves the eyes and remainder of the face (forehead, etc) free.

It can be seen that the apparatus and method are also applicable for full-face masks of the type that seal around the perimeter of the face, or for a mask of the type that only covers the eyes, such as a diving mask or swimming goggles. The apparatus and method is also suitable for mask such as sleep apnea masks, with the tool used to identify the geometry of a closed three-dimensional curve on a person's face where the mask will pass over their nose, cheeks, philtrum and upper lips, and around the nostrils.

1-28. (canceled)

29. A method for forming a belt made of a continuous closed loop of material for a face mask, comprising the steps of:
i) using a measuring means to trace the contours of the face of a user;
ii) using the data collected by the measuring means to create a closed 3D curve cut line on the belt having a minimum thickness of 3 mm;
iii) cutting the belt on its width along the cutline, the rear edge of the belt can be cut to match the curvature and contour of the face, in which the belt has a minimum width of 5 mm at any point along the belt;
iv) connecting the belt to a face mask body so that the belt forms a seal for the mask,
wherein between steps i) and ii) the method further comprises using the data collected by the measuring means to create a 3D model of the face as a mathematical representation, and extracting a closed 3D curve from the 3D model;

30. A method as claimed in claim 29, in which in the step of using a measuring means to trace the contours of the face of a user, a software program is used.

31. A method as claimed in claim 29, further comprising between steps i) and ii) a software program is used to analyse the data, and using the software program to mathematically convert the closed 3D curve to a 2D open curve.

32. A method as claimed in claim 29, wherein in the step of using a measuring means to trace the contours of the face of a user, an electronic measuring tool is used.

33. A method as claimed in claim 29, in which in step iii) the method comprises placing a belt on a cutting machine and transferring data to the cutting machine, so that the cutting machine cuts the belt in accordance with the 3D curve.

34. A belt for a face mask, comprising:
a loop of soft material suitable for extended skin contact and configured to connect to a mask body in use, the loop formed with a profile that generally matches that of the facial features in the location in which the face mask is to be used, the loop and ask body mutually configured to form a closed volume in front of at least a portion of the face of a user, the loop in use locating against the face of a user to form a seal;
the belt formed from the loop using the method of claim 29.

35. A belt for a face mask as claimed in claim 34, in which the loop is formed from neoprene, silicone rubber, elastic or soft polymer.

36. A face mask comprising:
a body comprising a substantially flat front face portion, with
a surrounding wall extending from the edge of the front face portion; and
a belt as claimed in claim 34,
in which the body and belt are configured for push or friction fit engagement.

37. An apparatus for customising the fit of a face mask, comprising:
a bar support structure configured to form a closed or substantially closed loop;
a plurality of elongate bars, the bars and bar support structure configured so that the bars are held in place on the bar support structure adjacent to one another around the closed loop;
the bar support structure further configured so that each of the individual bars can slide within the support structure in a direction substantially parallel to the longitudinal axis of the bar, at least one end of each bar protruding freely when the bar is extended within the support structure;
the closed loop of the bars sized and configured so as to form a loop of substantially equivalent size to the perimeter of a face mask,
wherein each of the bars comprises an elongate body and head, the head wider than the body, the bars and support structure configured so that the heads are substantially directly adjacent to one another.

38. A method of forming a belt for a face mask, comprising the steps of:
i) placing an apparatus as claimed in claim 37 against a face of a user, with the bars substantially fully extended;
ii) pressing the apparatus against the face of a user so that the bars slide inwards to form an overall shape that conforms to the contours of a user's face around a closed loop in the required location;
iii) positioning a belt comprising a continuous closed loop of soft material suitable for extended skin contact within the closed loop of the bars;
iv) tracing a line around the outer ends of the bars on the outer surface of the belt;
v) removing the belt and cutting along the traced line;
vi) connecting the belt to a face mask body so that the belt forms a seal for the mask.
wherein between steps iv) and v) of wrapping a strap or similar around the bars to hold these in place.
